(19) United States
(12) Patent Application Publication
(10) Pub. No.: US 2006/0099652 A1
(43) Pub. Date: May 11, 2006

(54) IL 13 receptor alpha 2 antibody and methods of use

(75) Inventors: Stephen T. Gately, Palatine, IL (US); Stephen P. Wanaski, Chicago, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL (US)

(21) Appl. No.: 11/220,888

(22) Filed: Sep. 7, 2005

Related U.S. Application Data
(63) Continuation of application No. PCT/US04/09354, filed on Mar. 26, 2004.
(60) Provisional application No. 60/457,898, filed on Mar. 26, 2003.

(51) Int. Cl. G01N 33/567; C07K 16/28; C07K 16/46

(52) U.S. Cl. 435/7.2; 530/388.22

(57) ABSTRACT

The invention provides an antibody directed against an IL13 receptor alpha 2 (IL13-Rα2) and methods of using the antibody to detect and localize IL13-Rα2, to diagnose a disease characterized by expression of IL13-Rα2, and to kill a cell that expresses an IL13-Rα2.

### CROSS-REFERENCE TO RELATED APPLICATIONS

This patent application is a continuation of International patent application No. PCT/US2004/009354, filed Mar. 26, 2004, which designated the United States, and which, in turn, claims priority to provisional patent Application 60/457,898, filed Mar. 26, 2003, the contents of which are hereby incorporated herein in their entirety by reference.

### FIELD OF THE INVENTION

This invention pertains to an IL13 receptor alpha 2 (IL13-Rα2) antibody and methods of using IL13-Rα2 antibodies.

### BACKGROUND OF THE INVENTION

Malignant glioma, including glioblastoma multiforme (GBM) and anaplastic astrocytoma (AA), occurs in approximately 17,500 patients annually in the United States. Despite an aggressive multimodal approach to its treatment, no curative therapy is known. Median survival expectation is 9-12 months from diagnosis for GBM and 24-48 months for AA. Despite numerous investigational trials, patients with a recurrence of malignant glioma after initial radiotherapy do not live long.

One approach to eradicating tumor cells is to target cytotoxic agents to the cells. To accomplish this, antibodies or growth factors that bind to cells can be attached to cytotoxic molecules. The binding sites on such cells are known as cell receptors. This method is selective in situations where the targeted receptors are present in substantially higher amounts on target cells than in normal cells. Selectivity is desirable as it minimizes toxicity to normal cells. Exceptionally high levels of the alpha 2 receptor for Interleukin 13 (IL13-Rα2) have been identified in a number of tumor cells, including malignant gliomas. In contrast, only a few types of normal cells express IL13-Rα2 and only at low levels. Consequently, antibodies that bind IL13-Rα2 have the potential to be an effective tool for the diagnosis, screening, and treatment of diseases associated with the expression of IL13-Rα2 on cell surfaces.

In this regard, overexpression of the IL13-Rα2 in a target (i.e., tumor) cell may predict a positive response to a therapeutic agent that targets IL13-Rα2. Moreover, localization of IL13-Rα2 expression to a particular cell or tissue type will allow physicians to more precisely identify those tissues affected by an IL13-Rα2-associated disease. In addition to diagnostic and preventative applications, antibodies or growth factors (i.e., IL13) that bind IL13-Rα2, when combined with a cytotoxic agent, also have the potential to be a highly effective therapeutic agent for the treatment of IL13-Rα2-expressing tumor cells. Despite the potential for such useful applications, currently each is hindered by difficulty in detecting IL13-Rα2 expression. Thus, there remains a need for compositions and methods that can be used to reliably and efficiently detect and localize IL13-Rα2 expression in cells, particularly tumor cells.

### BRIEF SUMMARY OF THE INVENTION

The invention provides an isolated antibody or antigen-binding fragment thereof directed against an IL13-Rα2 that binds an epitope comprising or consisting essentially of an amino acid sequence of SEQ ID NO:1. The invention also provides a method for detecting or localizing an IL13-Rα2 polypeptide in vitro comprising (a) contacting a sample or cell suspected of containing the IL13-Rα2 with an isolated antibody that binds the IL13-Rα2, and (b) detecting binding of the IL13-Rα2 antibody to the IL13-Rα2. Another aspect of the invention provides a method for diagnosing a disease characterized by expression of an IL13-Rα2 comprising contacting a cell with an isolated antibody that binds the IL13-Rα2, wherein the detectable binding of the IL13-Rα2 antibody indicates expression of the IL13-Rα2, and the disease is diagnosed. Another aspect of the invention provides a method for killing a cell that expresses an IL13-Rα2 comprising contacting the cell with an isolated antibody that binds to the IL13-Rα2 and is conjugated to a cytotoxic agent, such that the IL13-Rα2 antibody binds the IL13-Rα2 and the cytotoxic agent contacts the cell, whereby the cell is killed. These and other advantages of the invention, as well as additional inventive features, will be apparent from the description of the invention provided herein.

### DETAILED DESCRIPTION OF THE INVENTION

In one aspect, the invention provides an isolated antibody or antigen-binding fragment thereof directed against IL13-Rα2. The full-length sequence of the IL13-Rα2 cDNA and protein are set forth as SEQ ID NOs: 3 and 4, respectively. Any antibody (or fragment thereof) that binds the IL13-Rα2 is suitable for use in the invention. In one embodiment, the antibody or antigen-binding fragment thereof binds an epitope comprising or consisting essentially of an amino acid sequence of SEQ ID NO:1.

Antibodies, also known in the art as immunoglobulins, are molecules having a specific amino acid sequence, by virtue of which they interact only with the antigen that induced their synthesis in cells of the lymphoid series (especially plasma cells), or with an antigen closely related to it. The term “antigen” refers to any molecule that can bind specifically to an antibody. An antigen that can induce antibody production is typically referred to in the art as an immunogen. Antibodies typically are produced in response to infection or immunization, bind to and neutralize pathogens, or prepare pathogens for uptake and destruction by phagocytes (see, e.g., C. A. Janeway et al. (eds.), Immunobiology, 5^{th}Ed., Garland Publishing, New York, N.Y. (2001)). The general structure and function of antibody molecules are well known in the art.

As used herein, an “isolated” antibody (or fragment thereof) refers to at least one antibody molecule (or fragment thereof) that has been isolated, or is otherwise free of, the bulk of the total antibodies circulating in the bloodstream of an animal. Total isolation from all other antibodies, however, is not necessary. Indeed, the inventive antibody composition can be polyclonal, in some embodiments. In other words, an antibody is “isolated” if it has been changed or removed from its natural in vivo environment.

Methods of generating antibodies using purified polypeptides or synthetic oligonucleotides are known in the art. Generally, such methods typically involve administering a polypeptide antigenic determinant (or an oligonucleotide encoding such an antigenic determinant) mixed with an adjuvant to an organism (e.g., a rabbit, mouse, sheep, etc.), such that antibodies directed against the antigen are produced by the organism (see, e.g., Harlow and Lane (eds.), Antibodies: A Laboratory Manual, CSH Press (1988), Salvatore et al., Biochem. Biophys. Res. Comm., 294, 813-817 (2002), and U.S. Pat. Nos. 5,776,457 and 5,614,191). Specific antibodies raised against the immunizing antigen can be isolated and purified from animal serum using any suitable method known in the art. Such methods include, for example, affinity chromatography, in which immunized serum is applied to beads loaded in a column that are covalently bound to the antigen of interest. Non-specific antibodies and other serum proteins are washed away, leaving only antigen-specific antibodies bound to the antigen coated beads, which are eluted by adjusting the pH, temperature, or salt concentration of the reaction conditions. Other suitable methods for antibody isolation and purification are disclosed in, for example, Published U.S. patent application No. 20020197266/A1, U.S. Pat. No. 5,776,457, and Janeway et al., supra.

While the inventive antibody (and composition comprising the same) preferably comprises an antibody directed against an IL13-Rα2, antibody fragments that recognize and bind one or more antigens of an IL13-Rα2 also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Alternatively, cleavage of an antibody molecule with the enzyme pepsin normally produces two antibody fragments, one of which retains both antigen-binding arms of the antibody molecule, and is thus referred to as the F(ab′)₂ fragment. Alternatively, a single-chain Fv antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Antibody fragments of the present invention, however, are not limited to these exemplary types of antibody fragments. Any suitable antibody fragment that recognizes and binds IL13-Rα2 is within the scope of the present invention. Antibody-antigen binding can be assayed using any suitable method known in the art, such as, for example, radioimmunoassay (RIA), enzyme-linked immunosorbent assay (ELISA), Western blot, immunoprecipitation, and competitive inhibition assays (see, e.g., Janeway et al., supra, and Published U.S. patent application No. 20020197266/A1).

Antibodies (or antibody fragments) that bind an IL13-Rα2 produced in accordance with the methods disclosed herein can be polyclonal antibodies (or antibody fragments), or monoclonal antibodies (or antibody fragments). As used herein, “polyclonal” antibodies (or antibody fragments) refer to heterogeneous populations of antibody molecules (or antibody fragments), typically obtained from the sera of immunized animals. “Monoclonal” antibodies (or antibody fragments) refer to homogenous populations of antibody molecules (or antibody fragments) that are specific to a particular antigen. Monoclonal antibodies typically are produced by a single clone of B lymphocytes (“B cells”). Monoclonal antibodies (or antibody fragments) may be obtained using a variety of techniques known to those skilled in the art, including standard hybridoma technology (see, e.g., Kohler and Milstein, Eur. J. Immunol., 5, 511-519 (1976), U.S. Pat. Nos. 4,376,1 10 and 5,614,191, Published U.S. patent application No. 20021972666/A1, Harlow and Lane, supra, and Janeway et al., supra). In brief, the hybridoma method of producing monoclonal antibodies typically involves injecting any suitable animal, typically and preferably a mouse, with an antigen (i.e., an “immunogen”). The animal subsequently is sacrificed and B cells isolated from its spleen are fused with myeloma cells. A hybrid cell (i.e., a “hybridoma”) is produced, which proliferates indefinitely in vitro and continuously secretes high titers of an antibody with the desired specificity. Any appropriate method known in the art can be used to identify hybridoma cells that produce an antibody with the desired specificity. Such methods include, for example, ELISA, Western blot analysis, and radioimmunoassay. The population of hybridomas is screened to isolate individual clones, each of which secrete a single antibody species to the antigen. Because each hybridoma is a clone derived from fusion with a single B cell, all the antibody molecules it produces are identical in structure, including their antigen binding site and isotype. Monoclonal antibodies (or antibody fragments) also may be generated using other suitable techniques including EBV-hybridoma technology (see, e.g., Haskard and Archer, J. Immunol. Methods, 74(2), 361-67 (1984) and Roder et al., Methods Enzymol., 121, 140-67 (1986)), or bacteriophage vector expression systems (see, e.g., Huse et al., Science, 246, 1275-81 (1989)). To prepare monoclonal antibody fragments, recombinant methods typically are employed.

The inventive antibody (or fragment thereof) can be isolated from or produced in any animal that can be immunized against an antigen or antigenic determinant of an IL13-Rα2. In one embodiment, the antibody desirably is isolated from or produced in an avian species, such as a chicken. Not to adhere to any one particular theory, it is believed that, due to the evolutionary distance between avian species and mammals, avian antibodies react with more epitopes on a mammalian antigen, resulting in signal amplification, and exhibit reduced cross-reactivity with mammalian antibodies and proteins, reducing background effects in immunological assays. In another embodiment, preferably, the antibody is isolated from or produced in a mammal, more preferably a mouse, and most preferably a human. Methods for producing an antibody using animals such as chicken or mice are well known to those skilled in the art and are described herein. In particular, methods for producing polyclonal and monoclonal antibodies in avian species are described in, for example, WO 01/88162 and WO 00/29444. With respect to human antibodies, one of ordinary skill in the art will appreciate that polyclonal antibodies can be isolated from the sera of human subjects vaccinated or immunized with antigenic portions of an IL13-Rα2. Alternatively, human antibodies directed against an IL13-Rα2 can be generated by adapting known techniques for producing human antibodies in non-human animals such as mice (see, e.g., U.S. Pat. Nos. 5,545,806 and 5,569,825, and Published U.S. patent application No. 20020197266/A1).

One of ordinary skill in the art will appreciate that, while being the ideal choice for therapeutic and diagnostic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the mouse antibody. To circumvent these complications, the inventive antibody (or fragment thereof) preferably exhibits reduced recognition by the human immune system as compared to an analogous non-human antibody. Most preferably, the inventive antibody is not recognized as “foreign” by the human immune system. To this end, phage display can be used to generate the inventive antibody. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell (see, e.g., Janeway et al., supra, Huse et al., supra, and U.S. Pat. No. 6,265,150). Alternatively, monoclonal antibodies can be generated from mice that are transgenic for specific human heavy and light chain immunoglobulin genes. Such methods are known in the art and described in, for example U.S. Pat. Nos. 5,545,806 and 5,569,825, and Janeway et al., supra). Most preferably, the inventive antibody is a humanized antibody. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway et al., supra, and U.S. Pat. Nos. 5,585,089 and 5,693,761.

The inventive antibody (or antibody fragment) may be of any immunoglobulin isotype. The term “isotype,” as is used in the art, typically describes the class, subclass, light chain type and subtype of an antibody. One of ordinary skill in the art will appreciate that the five major human immunoglobulin isotypes are immunoglobulin M (i.e., IgM), IgD, IgG, IgA, and IgE, which are typically defined by the structure of the constant regions of the antibody heavy chain. The light chain of a human antibody molecule is typically classified in the art as either a lambda (λ) chain or a kappa (κ) chain. IgG antibodies can be subdivided further into four subtypes (i.e., IgG1, IgG2, IgG3, and IgG4), whereas IgA antibodies typically are subdivided into two subtypes (i.e., IgA1 and IgA2). In embodiments where the inventive antibody is a chicken antibody, the antibody is preferably of the IgY isotype, which is the main serum immunoglobulin in chicken. Chicken IgY antibodies also are referred to in the art as chicken IgG antibodies, as they are the functional equivalent of mammalian IgG in birds. Like mammalian IgG antibodies, chicken IgY antibodies consist of two light chains and two heavy chains, and can be enzymatically cleaved into Fab fragments. IgY can be isolated from serum or collected from the yolks of eggs produced by immunized hens (see, e.g., Warr et al., Immunol. Today, 16, 392-98 (1995) and Haak-Frendscho M., Promega Notes Magazine, 46, 11 (1994)).

The inventive isolated antibody, or antigen-binding fragment thereof, can be directed against the full-length IL13-Rα2 or a fragment thereof. The structure and function of IL13-Rα2 have been characterized and described in, for example, Caput et al., J. Biol. Chem., 271, 16921-16926 (1996). Most preferably, the inventive antibody binds an epitope of an IL13-Rα2 comprising an amino acid sequence of SEQ ID NO:1, or consisting essentially of this sequence. An “epitope,” also known in the art as an “antigenic determinant,” is a site or an amino acid sequence recognized by an antibody or an antigen receptor. The epitope recognized by the inventive antibody can be derived from a naturally occurring IL13-Rα2, or synthetically generated using routine recombinant DNA and protein technology (see, e.g., Sambrook et al., supra).

In preferred embodiments, the inventive antibody can recognize any epitope comprising a variant or homolog of the polypeptide set forth in SEQ ID NO:1. A variant of the polypeptide can include a polypeptide encoded by a nucleic acid sequence comprising one or more mutations (e.g., point mutations, deletions, insertions, etc.) from the nucleic acid sequence encoding a corresponding naturally occurring protein. By “naturally occurring” is meant that the protein can be found in nature and has not been synthetically modified. Where mutations are introduced in the nucleic acid sequence encoding the polypeptide, such mutations desirably will effect a substitution in the encoded protein whereby codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. In addition, a homolog of the polypeptide can be any peptide, polypeptide, or portion thereof, that is more than about 70% identical (preferably more than about 80% identical, more preferably more than about 90% identical, and most preferably more than about 95% identical) to the polypeptide at the amino acid level. The degree of amino acid identity can be determined using any method known in the art, such as the BLAST sequence database.

Using any of the methods described herein, one of ordinary skill in the art will appreciate that an animal can be immunized to produce antibodies specific for a particular antigen or epitope by administering a suitable composition comprising a polypeptide encoding the antigen or epitope to the animal. Alternatively, a gene transfer vector comprising a nucleic acid sequence encoding the antigen or epitope can be generated and administered to an animal using any suitable method known in the art, such that the antigen or epitope is produced within the animal, resulting in an antibody response against the antigen or epitope within the animal. In this regard, the inventive IL13-Rα2 antibody preferably recognizes an epitope that is encoded by a nucleic acid sequence comprising SEQ ID NO:2, or consisting essentially of this sequence. The inventive antibody also can be generated by immunizing an animal with a nucleic acid sequence that encodes an epitope comprising any variant, homolog, or functional portion of SEQ ID NO:1, as described previously herein.

An epitope of an IL13-Rα2 can be identified using any suitable method known in the art. In this regard, nucleic acid sequences encoding peptide fragments of full-length IL13-Rα2 can be cloned into recombinant expression vectors using standard molecular biology techniques (see, e.g., Sambrook et al., supra). Putative IL13-Rα2 epitopes can be tested for antigenicity against sera containing IL13-Rα2 antibodies (e.g., sera isolated from a patient suffering from malignant glioma) in vitro, or by administering an expression vector encoding a putative epitope to an appropriate laboratory animal and assaying for anti-IL13-Rα2 antibody production. Methods for epitope mapping are known in the art and are described in, for example, U.S. Pat. No. 5,747,240. Methods for isolating full-length IL13-Rα2 polypeptides are described in, for example, U.S. Pat. No. 5,919,456.

In another embodiment, the invention provides a method for detecting an IL13-Rα2 polypeptide in vitro comprising (a) contacting a sample or cell suspected of containing IL13-Rα2 with an isolated antibody or fragment thereof that binds IL13-Rα2, and (b) detecting binding of the IL13-Rα2 antibody to IL13-Rα2. Any antibody (or fragment thereof) that binds IL13-Rα2, examples of which are set forth herein, is suitable for use in the inventive composition. The inventive method desirably employs an isolated antibody, or antigen-binding fragment thereof, that is directed against the full-length IL13-Rα2 or a fragment thereof. Isolated antibodies (or antibody fragments) that bind IL13-Rα2 have been developed and are available from a variety of sources, such as Cell Sciences, Inc. (www.cellsciences.com), and are described in, for example, Published U.S. patent application No. 20020197266/A1 and David et al., Oncogene, 20, 6660-6668 (2001). Most preferably, the inventive method employs the IL13-Rα2 antibody (or antibody fragment) described herein, i.e., an antibody (or antibody fragment) which binds an epitope of an IL13-Rα2 comprising an amino acid sequence of SEQ ID NO:1, or consisting essentially of this sequence.

In one aspect of the inventive method a sample or cell suspected of containing IL13-Rα2 is contacted with an isolated antibody or fragment thereof that binds IL13-Rα2, and binding of the IL13-Rα2 antibody to IL13-Rα2 is detected. The sample or cell suspected of containing IL13-Rα2 can be isolated or derived from any tissue, organ, fluid (e.g., blood, lymph, or serum), or the like, from any suitable animal. A sample or cell is “derived” from a source when it is isolated from a source but modified in any suitable manner (e.g., by introduction of exogenous nucleic acid sequences, or modification of endogenous genomic DNA) so as not to disrupt the normal function of the source sample or cell. Thus, one of ordinary skill in the art will appreciate that the inventive method can be used to determine expression of IL13-Rα2 in a sample or cell at the cellular or subcellular level, as well as the presence of soluble forms of IL13-Rα2 in a liquid sample (e.g., bodily fluid). The sample or cell preferably is isolated or derived from a mammal, most preferably a human. The sample or cell preferably is either a tissue sample isolated or derived from a mammal or is a cell grown in cell culture. In a particularly preferred embodiment, the sample or cell is isolated or derived from an organ, tissue, fluid, or the like, that is suspected to be affected by any disease caused by or associated with expression of IL13-Rα2. In this regard, the sample or cell preferably is isolated or derived from a solid tumor, such as, for example, an organ or tissue affected by malignant glioblastoma multiforme (GBM), anaplastic astrocytoma (AA), Kaposi sarcoma (KS), and renal cell carcinoma (RCC). The inventive method, however, is not limited to detecting IL13-Rα2 expression in these exemplary tumor types. The inventive method can be practiced using any sample or cell suspected of containing (i.e., expressing) an IL13-Rα2.

The sample or cell is contacted with an antibody (or antibody fragment) that binds IL13-Rα2 using any suitable method known in the art. Such methods can be in vitro or in vivo. Suitable in vitro methods for contacting the sample or cell include, include, for example, providing the antibody (or antibody fragment) to the culture medium in which the sample or cell is maintained or propagated. Alternatively, the antibody (or antibody fragment) can be provided by transfecting a culture of cells suspected of containing IL13-Rα2 with an expression vector comprising a polynucleotide sequence encoding the antibody (or antibody fragment), such that the polynucleotide is expressed and the antibody (or antibody fragment) is produced in the cell. In yet another alternative, lysates of cells suspected of containing IL13-Rα2 can be prepared using routine cell culture techniques and incubated with an antibody (or antibody fragment) that binds IL13-Rα2. With respect to in vivo contacting methods, one of ordinary skill in the art will appreciate that an antibody (or antibody fragment) can be formulated into a composition comprising a physiologically acceptable carrier and administered directly to an animal (e.g., a human) via numerous routes. Exemplary formulations, carriers, and administration routes for in vivo administration of an IL13-Rα2 antibody (or fragment thereof) are known in the art and described elsewhere herein. The present invention, however, is not limited to these exemplary in vitro and in vivo contacting methods. Any suitable method for contacting a sample or cell with an IL13-Rα2 antibody (or fragment thereof) is within the scope of the present invention.

Detecting binding of an IL13-Rα2 antibody to IL13-Rα2 can be performed using any suitable method to detect protein-protein, ligand-receptor, and/or antibody-antigen interactions. Such methods are well known to those skilled in the art, and include, for example, flow cytometry, ELISA, affinity chromatography, competitive inhibition assay, radioimmunoassay, immunofluorescence microscopy, immunoelectron microscopy, immunocytochemistry (also referred to in the art as immunohistochemistry), and immunoprecipitation. Such methods are described in, for example, Janeway et al., supra, David et al., supra, Salvatore et al., Biochem. Biophys. Res. Comm., 294, 813-817 (2002), Berger et al., Eur J Cell Biol., 67, 106-11 (1995), Cechetto et al., Exp Cell Res., 260, 30-39 (2000), Gruber et al., BMC Musculoskeletal Disorders, 3, 1-5 (2002), Harlow and Lane, supra, and U.S. Pat. Nos. 4,661,913, 5,366,859, 5,491,096, and 5,958,715. The inventive method, however, should not be construed as being limited to these exemplary detection methods. Indeed, any method that can detect binding of an IL13-Rα2 antibody to IL13-Rα2 is within the scope of the present invention. One of ordinary skill in the art will appreciate that these exemplary methods also can enable the quantification of the amount of IL13-Rα2 expressed in a particular sample or cell, as well as the actual number of IL13-Rα2 receptors present in a particular sample or cell. Thus, the aforementioned methods for detecting IL13-Rα2 expression in a sample or cell also can be employed to quantify the number of IL13-Rα2 polypeptides that are present in the sample or cell. Most preferably, the number of IL13-Rα2 polypeptides in a sample or cell is quantified using flow cytometry-based applications.

The present invention also provides a method for localizing IL13-Rα2 in a sample or cell comprising (a) contacting the sample or cell with an isolated antibody that binds the IL13-Rα2, (b) detecting binding of the IL13-Rα2 antibody to the IL13-Rα2, and (c) determining the location of the IL13-Rα2 in the sample or cell. Descriptions of the antibody (or antibody fragment), the sample or cell, the detection methods, and components thereof set forth above in connection with other embodiments of the invention also are applicable to those same aspects of the aforesaid inventive method.

Methods for determining the location of (i.e., localizing) a polypeptide (e.g., a receptor) within a cell are known in the art, some of which are identical to those described herein for detecting ligand-receptor or antigen-antibody binding. Thus, in some embodiments of the invention, methods that detect binding of an IL13-Rα2 antibody (or antibody fragment) to IL13-Rα2 also will reveal the location of the IL13-Rα2 within the cell. Such methods preferably include, for example, immunofluorescence microscopy, immunoelectron microscopy, and immunocytochemistry. While electron microscopy provides higher resolution, light microscopy can provide sufficient spatial resolution in less time, and also can be used in connection with the inventive method. In other embodiments, detection of the IL13-Rα2 antibody (or antibody fragment) binding to IL13-Rα2 will not provide any information with respect to the location of the IL13-Rα2 in a sample or cell. In such cases, therefore, localization of the IL13-Rα2 must be determined separately from, and following, the detection of an IL13-Rα2 antibody (or antibody fragment) binding to IL13-Rα2.

As mentioned herein, expression (or overexpression) of IL13-Rα2 is associated with a number of pathological conditions, including certain types of cancer. Thus, the present invention further provides a method for diagnosing a disease characterized by expression of IL13-Rα2 comprising contacting a cell with an isolated antibody that binds IL13-Rα2, wherein the detectable binding of the IL13-Rα2 antibody indicates expression of the IL13-Rα2, and the disease is diagnosed. In this respect, methods described herein for detecting IL13-Rα2 expression in a sample or cell also can be used in connection with the aforementioned method for diagnosing a disease. Moreover, descriptions of the antibody (or antibody fragment), the sample or cell, and components thereof set forth above in connection with other embodiments of the invention also are applicable to those same aspects of the aforesaid inventive method. The inventive method can be used to diagnose any disease associated with or caused by IL13-Rα2 expression. In a particularly preferred embodiment, the inventive method is used to diagnose cancer in a patient. In this regard, the sample or cell preferably is a tumor cell. Most preferably, the sample or cell is derived from a malignant glioma, such as, for example, glioblastoma or anaplastic astrocytoma. Alternatively, the inventive method can be used to diagnose other cancers associated with or caused by IL13-Rα2 expression, such as, for example, Kaposi sarcoma (KS) or renal cell carcinoma (RCC).

The invention provides a method for killing a cell that expresses IL13-Rα2 comprising contacting the cell with an isolated antibody that binds to IL13-Rα2 and is conjugated to a cytotoxic agent, such that the IL13-Rα2 antibody binds IL13-Rα2 and the cytotoxic agent contacts the cell, whereby the cell is killed. Descriptions of the antibody (or antibody fragment), and components thereof, set forth above in connection with other embodiments of the invention also are applicable to those same aspects of the aforesaid inventive method. In this embodiment, the IL13-Rα2 antibody (or antibody fragment) can be used as a therapeutic agent to target and kill cells that express or overexpress an IL13-Rα2. Suitable target cells have been described herein, and include tumor cells such as malignant glioma cells (e.g., glioblastoma and anaplastic astrocytoma), Kaposi's sarcoma cells, and renal cell carcinoma cells. The inventive method, however, is not limited to these exemplary target cells. Indeed, cells derived from tissue affected by any disease associated with or caused by IL13-Rα2 expression can be targeted and killed in accordance with the inventive method, thereby preferably resulting in treatment of the disease. In an alternative embodiment, the IL13-Rα2 antibody can be used to treat conditions associated with IL13-induced inflammation, such as, for example, certain allergic conditions including asthma. In this regard, the inventive IL13-Rα2 antibody can be used as therapeutic agent to bind IL13-Rα2, thereby preventing IL13 from binding to the receptor. In this manner, signaling through the IL13-Rα2 is blocked, and IL13-mediated inflammation is inhibited.

The isolated antibody (or antibody fragment) preferably is conjugated to a cytotoxic agent. Any suitable cytotoxic agent that can be joined to the IL13-Rα2 can be used in practicing the present invention, so long as sufficient cytotoxicity is preserved in the ultimate conjugate molecule. The IL13-Rα2 antibody (or antibody fragment) and cytotoxic agent can be joined by any suitable means that provides for retention of the targeting and cytotoxicity characteristics of the IL13-Rα2 antibody (or antibody fragment) and cytotoxic agent, respectively. For example, the IL13-Rα2 antibody (or antibody fragment) and cytotoxic agent can be joined chemically such as through cysteine disulfide or other chemical conjugation methods. Desirably, the IL13-Rα2 antibody (or antibody fragment) and cytotoxic agent are joined at the genetic level in a recombinant fusion protein, such as is described in U.S. Pat. Nos. 5,614,191 and 5,919,456.

Many cytotoxic molecules are known and are suitable for use as the cytotoxic agent. Suitable toxins include Pseudomonas exotoxin, ricin, Diphtheria toxin, abrin, a radionuclide (i.e., a radioisotope), and the like. Suitable cytotoxic agents maintain their cytotoxicity when joined with the IL13-Rα2 antibody. Derivatives of the cytotoxic agent, including genetic and chemical derivatives, are also suitable for use so long as sufficient cytotoxicity is preserved in the ultimate antibody-cytotoxic agent conjugate.

In a preferred embodiment of the invention, the IL13-Rα2 antibody (or antibody fragment) is introduced to human cells in vivo. In such applications, the method can be used alone or adjunctively as part of a treatment for any of a number of malignancies, such as those set forth above. For use in vivo, the antibody (or antibody fragment) desirably is formulated into a composition comprising a physiologically acceptable carrier. Any suitable physiologically acceptable carrier can be used within the context of the invention, and such carriers are well known in the art.

The carrier typically will be liquid, but also can be solid, or a combination of liquid and solid components. The carrier desirably is physiologically acceptable (e.g., a pharmaceutically or pharmacologically acceptable) carrier (e.g., excipient or diluent). Physiologically acceptable carriers are well known and are readily available. The choice of carrier will be determined, at least in part, by the location of the target tissue and/or cells, and the particular method used to administer the composition. In terms of using polypeptide therapeutics as active ingredients, the technology of U.S. Pat. Nos. 4,608,251, 4,601,903, 4,559,231, 4,559,230, and 4,596,792, each incorporated herein by reference, can be used.

Typically, such compositions can be prepared as injectables, either as liquid solutions or suspensions; solid forms suitable for using to prepare solutions or suspensions upon the addition of a liquid prior to injection can also be prepared; and the preparations can also be emulsified. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

The antibody for use in the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described. In a preferred embodiment of the invention, the IL13-Rα2 antibody (or antibody fragment) is formulated for injection or parenteral administration. In this regard, the formulation desirably is suitable for intratumoral administration, but also can be formulated for intravenous injection, intraperitoneal injection, subcutaneous injection, and the like.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the IL13-Rα2 antibody (or antibody fragment) and physiological distress. Immune system suppressors can be administered with the composition method to reduce any immune response to the antibody itself or associated with a disorder. Alternatively, immune enhancers can be included in the composition to upregulate the body's natural defenses against disease. Moreover, cytokines can be administered with the composition to attract immune effector cells to a disease (e.g., tumor) site.

The following examples further illustrate the invention but, of course, should not be construed as in any way limiting its scope.

### EXAMPLE 1

This example demonstrates the generation of an isolated antibody directed against IL13-Rα2 that binds an epitope comprising an amino acid sequence of SEQ ID NO:1.

Immunogenic epitopes of the IL13-Rα2 receptor were identified using DNA sequence analysis and epitope mapping techniques known in the art and described herein. A nucleic acid sequence of SEQ ID NO:2 was identified as encoding an IL13-Rα2 epitope comprising an amino acid sequence of SEQ ID NO:1. An expression vector comprising SEQ ID NO:2 operatively linked to a CMV promoter was generated as described in WO 00/29444. Chickens of strain Hy-line SC (Hyline, Inc., Dallas Center, Iowa) were vaccinated by administration of the expression vector to chicken back skin using gene gun technology known in the art (see, e.g., WO 00/29444 and WO 01/88162).

Twenty days post immunization, 10 eggs from each immunized chicken were collected for antibody isolation. In this regard, IgY antibodies specific for the IL13-Rα2 epitope comprising SEQ ID NO:1 were isolated from egg yolks and purified as described in Polson et al., Immunol. Commun., 9, 475-493 (1980) and in WO 01/88162 and WO 00/29444.

### EXAMPLE 2

This example demonstrates the detection and localization IL13-Rα2 in a sample using the antibody of Example 1.

U251 human glioblastoma cells and normal control brain cells are cultured under standard conditions and metabolically labeled with [³⁵S] methionine as described in Harlow and Lane, supra. Cell lysates are prepared in and incubated with the antibody of Example 1. Beads coated with protein A purified from S. aureus, which binds to the Fc portion of an antibody, are added, and the beads are collected via centrifugation. In this manner, collection of the protein A beads results in purification of any antigen-antibody complexes (“immunoprecipitates”) that have formed. The immunoprecipitates are washed and separated by SDS polyacrylamide gel electrophoresis (SDS-PAGE) using methods known in the art. The gel is dried and visualized via autoradiography. Immunoprecipitation methods are described in detail in Harlow and Lane, supra.

Localization of IL13-Rα2 is performed using immunocytochemistry methods known to those skilled in the art. Briefly, U251 cells and control cells are fixed with formalin, and tissue sections are prepared. Tissue sections are incubated with the antibody of Example 1. The cells are washed with PBS and incubated with an anti-chicken secondary antibody conjugated to biotin. To detect biotinylated antibodies, the sections are incubated with streptavidin that is either fluorescently labeled or conjugated to a calorimetric enzyme, such as horseradish peroxidase. Antibody binding is visualized via fluorescence microscopy or light microscopy, depending on the secondary antibody used. Standard immunocytochemistry techniques are described in detail in, for example, Janeway et al., supra, and Gruber et al., supra.

### EXAMPLE 3

This example demonstrates a method of killing a cell that expresses IL13-Rα2 comprising contacting the cell with an IL13-Rα2 antibody that is conjugated to a cytotoxic agent.

A fusion protein comprising the IL13-Rα2 antibody of Example 1 and a mutated and truncated form of Pseudomonas exotoxin is generated as described herein using standard molecular biology techniques (see, e.g., Sambrook et al., supra). Intratumoral injections of the antibody-exotoxin conjugate in concentrations of 50 and 100 μg/kg/day are administered for five consecutive days into nude mice having subcutaneous U251 glioblastoma tumors, resulting in a complete response (eradication of the tumor). Three alternate day intratumoral injections of the antibody-exotoxin conjugate at a dose of 250 μg/kg/day into subcutaneous U87 glioblastoma tumors also produce a complete response in all mice.

A 25 or 50 μg/kg/dose of the antibody-exotoxin conjugate is administered to nude mice having U251 xenografts via intraperitoneal injection for five days, twice daily, resulting in tumor regression or complete response. A 50 μg/kg intraperitoneal injection into nude mice having U87 xenografts causes a reduction in the tumor burden to one-half. Likewise, daily intravenous injections of the antibody-exotoxin conjugate at doses of 25 and 50 μg/kg for five days suppresses the growth of subcutaneous U251 tumors or results in a complete response in the animals of each treatment group. The antibody-exotoxin treatment manifests no toxicity in any of the treated mice.

The IL13-Rα2 antibody-exotoxin conjugate is directly injected into glioblastoma multiforme tumors xenografted into the right caudate nucleus of nude rat brain. A single injection of 33.3 μg/kg of antibody-exotoxin conjugate into intracranial tumors increases the median survival by >20% compared to control rats.

### EXAMPLE 4

This example demonstrates the sensitivity of the IL13-Rα2 antibody described in Example 1.

Evaluation of sensitivity of the test was performed on a combination of 29 human astrocytoma and renal cell carcinoma specimens. Formalin-fixed, paraffin-embedded tissues were used in this indirect IHC test using the biotinylated rabbit anti-IgY secondary antibody and the streptavidin-HRP tertiary reagent. Positive membranous and cytoplasmic staining is expected in tumor cells of human astrocytoma and renal cell carcinoma specimens, based on literature reports (see the list of citations at the conclusion of this Specification).

The immunohistochemistry was performed by first de-parrafinizing and re-hydrating the sections in graded alcohols. Heat-induced epitope retrieval then was performed using BORG buffer, 3 min., at 120 ° C., 20 psi, followed by Trypsin (0.025% Trypsin in PBS), 1 minute. Following this treatment, the sections were blocked with perodxidase in 3% H₂O₂ in PBS for 15 minutes at room temperature. The sections then were washed 3 times for 3 minutes in TBST. Then, the sections were exposed to the anti IL1-3Rα2 IgY at 0.5 mg/ml (diluted in DAKO diluent), for 30 minutes at room temperature. Following exposure to the antibody, the sections were again washed 3 times for 3 minutes each in TBST. Next, the sections were blocked with a 5% blocking solution (in PBS) for 10 minutes at room temperature, which was tapped off prior to exposure to secondary antibody. The sections then were exposed to a biotinylated rabbit anti-IgY secondary antibody (GenWay Biotech) at 10 mg/ml (diluted in DAKO diluent), for 15 minutes at room temperature. Following exposure to the secondary antibody, the sections were again washed 3 times for 3 minutes each in TBST. Next, the sections were exposed to streptavidin peroxidase (Pierce Chemical Co., Rockford, Ill.) at 0.5 U/ml (diluted in DAKO diluent), for 20 minutes at room temperature. Following this treatment, the sections were again washed 3 times for 3 minutes each in TBST. The sections then were treated with DAB (DakoCytomation, Carpinteria, Calif.) for 5 minutes at room temperature, following which, they were washed in deionized H₂O. Following the wash, the sections then were counterstained, dehydrated, and cover-slipped and observed via microscopy.

Adequate sensitivity was demonstrated by positive staining of the antibody in tumor cells of the astrocytoma and renal cell carcinoma specimens (see table 1). The acceptance criterion for the highest staining intensity is defined as greater than or equal to 10% of tumor cells staining positive at the specified intensity. Strong (3+) membranous and cytoplasmic staining was observed in 31% (9/29) of astrocytoma and renal cell carcinoma specimens, and moderate (2+) staining in 13.8% (4/29) of the specimens. Weak (1+) membranous and cytoplasmic staining was seen in 48.3% (14/29) of astrocytoma and renal cell carcinoma specimens. No staining was observed in 6.9% (2/29) of the specimens. The sensitivity of the test, shown as percentage of positive staining in astrocytoma and renal carcinoma specimens, is consistent with the expression of IL13-Rα2 in astrocytoma and renal cell carcinoma, based on literature reports (see the list of citations at the conclusion of this Specification).

TABLE 1
Atibody Validation
Sesnsitivity Worksheet

| | |
|---|---|
| Study Number: | NPH13 |
| Antibody Name: | Affi-Auti-IL-I3Rα2 IgY |
| Clone Name: | N/A |
| Manufacturer: | GenWay Biotech, Inc. |
| Reviewer: | J. Hiserodt, M.D., Ph.D. |
| Target: | IL-13Rα2 |
| Working Conc.: | 10 μg/ml |
| Pretreatment: | HIER (BORG) 120° C., 3′ + Trypsin 1′ |
| Isotype: | Chicken IgY |

| | | | | | Distinctive Tissue Element | | | | | | | | | | | | | |
|---|---|---|---|---|---|---|---|---|---|---|---|---|---|---|---|---|---|---|
| | | | | | (Endothelium) | | | | | Long | | | | | | | | |
| General Specimen Information | | | | | % Cells Staining at Each Intensity | | | | | H- | | | Smooth | | Inflam. | | | Date |
| Specimen ID | Tissue Type | Background | 3+ | Sub | 2+ | Sub | 1+ | Sub | 0 | Score | Normal | Endothelium | Muscle | Fibroblast | Stroma | Cells | Nerve | Stained |

Controls

| | | | | | | | | | | | | | | | | | | | |
|---|---|---|---|---|---|---|---|---|---|---|---|---|---|---|---|---|---|---|---|
| C | ITKI02476B | Renal Cell CA | 0 | 30 | M, C | 40 | M, C | 30 | M, C | 0 | 200 | NS | ± | NS | 2± | ± | 0 | NS | May 2, 2003 |
| C | ITCC04367D | AI72 Cell Line | 0 | 90 | C | 10 | C | 0 | | 0 | 290 | NA | NA | NA | NA | NA | NA | NA | May 2, 2003 |

Samples

| | | | | | | | | | | | | | | | | | | | |
|---|---|---|---|---|---|---|---|---|---|---|---|---|---|---|---|---|---|---|---|
| 1 | ITGL0107-185-00852-02 | c/w Glioblastoma | 0 | 0 | | 0 | | 70 | C | 30 | 70 | NS | 0 | NS | NS | ± | 0 | NS | May 2, 2003 |
| 2 | ITGL0110-307-00779-02 | c/w Astrocytoma¹ | 0 | 0 | | 0 | | 100 | C | 0 | 100 | NS | 0 | NS | NS | ± | 1+ | NS | May 2, 2003 |
| 3 | ITGL0110-307-02287-01 | c/w Astrocytoma | 0 | 0 | | 0 | | 100 | C, M² | 0 | 100 | NS | ± | NS | NS | ± | NS | NS | May 2, 2003 |
| 4 | ITGL0110-307-02291-02 | c/w Astrocytoma | 0 | 0 | | 5 | C | 95 | C | 0 | 105 | NS | ± | NS | NS | ± | NS | NS | May 2, 2003 |
| 5 | ITGL0110-307-02297-01 | c/w Astrocytoma | 0 | 15 | C, M | 30 | C, M | 55 | C | 0 | 160 | NS | NS | NS | NS | ± | NS | NS | May 2, 2003 |
| 6 | ITGL0110-307-02298-01 | c/w Astrocytoma | 0 | 0 | | 0 | | 100 | C³ | 0 | 100 | 3+ C⁴ | 3+ | NS | NS | ± | NS | NS | May 2, 2003 |
| 7 | ITGL0110-307-02300-02 | c/w Astrocytoma | 0 | 2 | M, C | 3 | M, C | 95 | C | 0 | 107 | NS | 0 | NS | NS | 0 | NS | NS | May 2, 2003 |
| 8 | ITGL0202-307-02141-01 | c/w Astrocytoma | 0 | 0 | | 0 | | 0 | | 100 | 0 | NS | 1+ | NS | NS | ± | NS | NS | May 2, 2003 |
| 9 | ITGL0202-307-02143-06 | c/w Astrocytoma | 0 | 5 | M, C | 5 | M, C | 90 | C | 0 | 115 | 0-1+ C | 1+ | NS | NS | ± | NS | NS | May 2, 2003 |
| 10 | ITGL0205-307-00709-02 | c/w Astrocytoma | 0 | 0 | | 10 | C, M | 80 | C | 10 | 100 | NS | 0 | 1-2+ | NS | 1+ | NS | NS | May 2, 2003 |
| 11 | ITGL0205-292-00341-02 | c/w Astrocytoma | 0 | 0 | | 10 | C | 60 | C | 30 | 80 | NS | 0 | NS | NS | ± | NS | NS | May 2, 2003 |
| 12 | ITGL0110-307-00781-1 | c/w Astrocytoma | 0 | 15 | M, C | 50 | C, M | 35 | C | 0 | 180 | NS | 0-± | NS | NS | ± | NS | NS | May 2, 2003 |
| 13 | ITGL0204-307-00786-1 | c/w Astrocytoma | 0 | 0 | | 0 | | 100 | C | 0 | 100 | NS | 0-± | NS | NS | ±-1+ | 0 | NS | May 2, 2003 |
| 14 | ITGL0205-292-00954-4 | c/w Astrocytoma | 0 | 20 | C, M | 30 | C, M | 50 | C | 0 | 170 | 0-1+ C | 0-1+ C | NS | NS | 1+ | NS | NS | May 2, 2003 |
| 15 | ITKI02472A | c/w Renal Cell CA | 0 | 5 | M, C | 5 | M, C | 90 | C | 0 | 115 | NS | 1+ | NS | 2+ | ± | NS | NS | May 2, 2003 |
| 16 | ITKI02474A | Renal Cell CA | 0 | 10 | M, C | 20 | M, C | 70 | C, M | 0 | 140 | NS | 1+ | NS | NS | ± | 0 | NS | May 2, 2003 |
| 17 | ITKI02475A | Renal Cell CA | 0 | 50 | C | 30 | C | 20 | C | 0 | 230 | NS | 0 | NS | NS | ± | 0 | NS | May 2, 2003 |
| 18 | ITKI02477A | Renal Cell CA | 0 | 30 | C, M | 20 | C, M | 50 | C, M | 0 | 180 | NS | 1+ | NS | NS | ± | NS | NS | May 2, 2003 |
| 19 | ITKI02479A | Renal Cell CA | 0 | 10 | C | 40 | C | 50 | C | 0 | 160 | NS | 1-2+ | NS | NS | ± | NS | NS | May 2, 2003 |
| 20 | ITKI02480A | Renal Cell CA | 0 | 0 | | 0 | | 100 | C, M | 0 | 100 | NS | 1-2+ | NS | NS | 0 | NS | NS | May 2, 2003 |
| 21 | ITKI02481A | Renal Cell CA | 0 | 0 | | 5 | M, C | 95 | M, C | 0 | 105 | NS | 1+ | 1-2+ | NS | 0 | NS | NS | May 2, 2003 |
| 22 | ITKI02482A | Renal Cell CA | 0 | 20 | C | 50 | C | 30 | C | 0 | 190 | NS | 0-1+ | 1-2+ | NS | 0 | NS | NS | May 2, 2003 |
| 23 | ITKI02542A | Renal Cell CA | 0 | 10 | C | 20 | C | 70 | C | 0 | 140 | NS | 0-1+ | NS | NS | 0 | NS | NS | May 2, 2003 |
| 24 | ITKI02544A | Renal Cell CA | 0 | 0 | | 5 | M | 95 | M | 0 | 105 | NS | 1-2+ | 1+ | NS | 0 | NS | NS | May 2, 2003 |
| 25 | ITKI02547A | Renal Cell CA | 0 | 0 | | 5 | M, C | 95 | C | 0 | 105 | NS | 1-2+ | NS | 2+ | 0 | NS | NS | May 2, 2003 |
| 26 | ITKI02548A | Renal Cell CA | 0 | 0 | | 2 | C | 98 | M, C | 0 | 102 | NS | 1+ | NS | NS | ± | NS | NS | May 2, 2003 |
| 27 | ITKI02549A | Renal Cell CA | 0 | 0 | | 0 | | 100 | M | 0 | 100 | NS | 2+ | NS | NS | ± | NS | NS | May 2, 2003 |
| 28 | ITKI03601A | c/w Renal Cell CA | 0 | 0 | | 0 | | 0 | | 100 | 0 | NS | 0 | NS | ± | ± | NS | NS | May 2, 2003 |
| 29 | ITKI03621A | Renal Cell CA | 0 | 0 | | 5 | M, C | 85 | M | 10 | 95 | NS | 1-2+ | 2+ C | NS | ± | NS | NS | May 2, 2003 |

Totals

| | |
|---|---|
| Number of samples staining 3+ and >9% | 9 |
| Number of samples staining 0 at 100% | 2 |
| Average H-Score of samples | 115.7 |

Comments:
¹Tissue is mostly necrotic. Only small focus of viable tumor.
²Unusual cytoplasmic granular staining
³Very weak staining
⁴Choroid plexus
± = Equivocal Results
NA = Not Applicable
NS = Not Seen
A = Apical Staining
B = Basal Layer Staining
C = Cytoplasmic Staining
F = Focally Positive
H = Heterogencous Staining
I = Inflammatory Cells
La = Luminal Accentuation
M = Membrane Staining
N = Nuclear Staining
P = Perineural Staining
S = Stroma
Sc = Scattered
Sub = Subcellular Localization

### EXAMPLE 5

This example demonstrates the reactivity pattern of the IL13-Rα2 antibody described in Example 1.

Evaluation of the antibody reactivity pattern was performed in as selection of 30 normal human tissue types. The immunohistochemistry was performed as described in Example 4. For each tissue type, three specimens were tested. Positive membranous and cytoplasmic staining is expected in a variety of normal tissues, based on literature reports (see the list of citations at the conclusion of this Specification).

Differential staining of the antibody in a variety of normal human tissue specimens demonstrated adequate specificity (see Table 2). The acceptance criterion for the highest staining intensity is defined as greater than or equal to 10% of the distinctive tissue element staining positively at the specified intensity. Strong (3+) membranous and cytoplasmic staining was observed in tissue sections from: colon, kidney, and placenta. Moderate (2+) membranous and cytoplasmic staining was observed in tissue sections from bladder, bone marrow, breast, cervix, lung, lymph node, pancreas, parotid, pituitary, prostate, small intestine, stomach, testis, tonsil, ureter, and uterus. Weak (1+) cytoplasmic staining was observed in tissue sections from adrenal, esophagus, heart, liver, muscle, ovary, skin, spinal cord, and thyroid. Unusual coarse cytoplasmic granular straining was seen in adrenal, breast, cervix, colon, esophagus, kidney, pancreas, parotid, small intestine, stomach, thyroid, ureter, and uretus tissues. The significance of such staining is uncertain and the possibility of staining artifact cannot be excluded since coarse cytoplasmic staining is not characteristic for specific IHC staining of IL13Rα2. Furthermore, coarse cytoplasmic staining is not an uncommon staining artifact in IHC, generally. No staining was observed in brain and spleen. The staining pattern is consistent with expression of IL13Rα2 in normal tissues, based on literature reports (see the list of citations at the conclusion of this Specification).

TABLE 2
Atibody Validation
Specificity Worksheet

| | |
|---|---|
| Study No.: | NPH13 |
| Antibody: | Affi-Anti-IL-13Rα2 IgY |
| Clone: | Polyclonal, Affinity Purified |
| Manufacturer: | GenWay Biotech, Inc. |
| Target: | IL-13Rα2 |
| Working Conc.: | 10 μg/ml |
| Pretreatment: | HIER (BORG) 120° C. 3′ followed by trypsin 1′ |
| Isotype: | Chicken IgY |

| | | | | Staining of Distinctive | | | | | | | | | | |
|---|---|---|---|---|---|---|---|---|---|---|---|---|---|---|
| General Specimen Information | | | | Tissue Elements | | | | | | | | | | |
| | | | Isotype | % Cells Staining at | | | | Staining of Other Cell Types | | | | | | |
| | | Histological | Control | Each Intensity | | | | | Smooth | | | Inflam. | | Date |
| Specimen ID# | Tissue Type | Review | Background | 3+ | 2+ | 1+ | 0 | Endothelium | Muscle | Fibroblast | Stroma | Cells | Nerve | Stained |
| ITKI02476B | Kidney CA | Kidney CA | 0 | 20 M, C | 50 M, C | 30 C, M | 0 | 2+ C | 1+ C | NS | 0-± | 0-3+ M, C | NS | May 6, 2003 |
| ITCC04367D | A172 Cell Line | Cell Line | 0 | 50 C, M | 50 C | 0 | 0 | NA | NA | NA | NA | NA | NA | May 6, 2003 |
| ITKI02476B | Kidney CA | Kidney CA | 0 | 20 C, M | 40 C, M | 40 C, M | 0 | 0-± | ±-1+ | NS | ± | 0-2+ | NS | May 15, 2003 |
| ITCC04367D | A172 Cell Line | Cell Line | 0 | 0 | 20 C | 80 C | 0 | NA | NA | NA | NA | NA | NA | May 15, 2003 |
| ITKI02476A | Kidney CA | Kidney CA | 0 | 30 C, M | 40 C, M | 30 C, M | 0 | 0-1+ C | NS | NS | ± | 0 | NS | May 30, 2003 |
| ITCC04367D | A172 Cell Line | Cell Line | 0 | 0 | 60 C, M | 40 C, M | 0 | NA | NA | NA | NA | NA | NA | May 30, 2003 |
| ITKI02476B | Kidney CA | Renal Cell CA | 0 | 50 C, M | 20 C, M | 30 C, M | 0 | 0 | NS | 2+ C | ± | 0 | NS | Jun. 10, 2003 |
| ITCC04367D | A172 Cell Line | Cell Line | 0-1+ C | 0 | 50 C | 50 C | 0 | NA | NA | NA | NA | NA | NA | Jun. 10, 2003 |
| INAD02614A | Adrenal | c/w Adrenal | 0 | 0 | 5 C¹ | 60 C¹ | 35 | ± | 1-2+ C, F | NS | 0-1+ F | 0 | 0 | May 15, 2003 |
| INAD03644A | Adrenal | Adrenal | 1+ | 0 | 0 | 50 C¹ | 50 | ± | 2+ C | NS | ± | 0 | 2+ | May 15, 2003 |
| INAD03648A | Adrenal | Adrenal | 1+ | 0 | 0 | 70 C¹ | 30 | ± | 2+ C | NS | ± | NS | NS | May 15, 2003 |
| INBL01846A | Bladder | Bladder | 0 | 0 | 30 C | 70 C | 0 | ± | 2+ C | NS | ± | 1-2+ M, C | NS | May 15, 2003 |
| INBL02983A | Bladder | Bladder | 0 | 0 | 0 | 100 M, C | 0 | 0 | 2+ C | NS | 0 | NS | NS | May 15, 2003 |
| INBL02997A | Bladder | Bladder | 0 | 0 | 30 C, M | 70 C, M | 0 | 0-± | 2+ C | NS | 0 | NS | NS | May 15, 2003 |
| INBO0111-307-00273-5 | Bone Marrow | Bone Marrow | 0 | 5 M, C | 5 M, C | 5 M, C | 85 | NS | NS | NS | 0 | NS | NS | May 30, 2003 |
| INBO0103-212-01323-1 | Bone Marrow | Bone Marrow | 0 | 0 | 0 | 0 | 100 | NS | NS | NS | 0 | NS | NS | May 30, 2003 |
| INBO0107-305-02724-1 | Bone Marrow | Bone Marrow | 0 | 5 C | 2 C | 2 C | 91 | NS | NS | NS | ± | NS | NS | May 30, 2003 |
| INBA02029B | Brain | Brain (Cortex) | 0 | 0 | 0 | 5 C | 95 | 0 | NS | NS | 1+² | NS | NS | May 15, 2003 |
| INCL02032A | Cerebellum | Brain | 0 | 0 | 0 | 0 | 100³ | ± | NS | NS | 1+² | NS | NS | May 15, 2003 |
| | | (Cerebellum) | | | | | | | | | | | | |
| INCL02035A | Cerebellum | Brain | 0 | 0 | 0 | 0 | 100³ | ± | NS | NS | 1+² | NS | NS | May 15, 2003 |
| | | (Cerebellum) | | | | | | | | | | | | |
| INBR01201A | Breast | Breast | 0 | 0 | 0 | 100 C | 0 | NS | NS | NS | 0 | 0-2+ | NS | May 15, 2003 |
| INBR01204A | Breast | Breast | 0 | 0 | 80 C¹ | 20 C¹ | 0 | ± | NS | NS | 0 | 0-2+ | NS | May 15, 2003 |
| INBR03066A | Breast | Breast | 1+ C¹ | 0 | 100 C¹ | 0 | 0 | 1+ C | NS | NS | ± | 0-1+ | NS | Jun. 10, 2003 |
| INCE00324C | Cervix | Cervix | 0 | 0 | 0 | 100 M₄, C | 0 | 0 | 1+ | NS | 0 | NS | NS | May 15, 2003 |
| INCE02778A | Cervix | Cervix | 0 | 0 | 50 C | 50 C | 0 | ± | NS | NS | 0-± | 1-2+ M, C | NS | May 15, 2003 |
| INCE0103-212-01579-1A | Cervix | Cervix | 0-1+ C, F | 0 | 20 C¹ | 80 C, M | 0 | ± | 1-2+ C | NS | 0 | NS | NS | May 15, 2003 |
| INCO03165A | Colon | Colon | 0 | 0 | 30 C¹ | 70 C¹ | 0 | 0-± | 2+ C | NS | 0 | 0-3+ M, C | NS | May 15, 2003 |
| INCO03179A | Colon | Colon | 0 | 100 C¹ | 0 | 0 | 0 | 0-± | 1-2+ C | NS | 0 | 0-2+ M, C | NS | May 15, 2003 |
| INCO03181D | Colon | Colon | 0 | 0 | 80 C¹ | 20 C¹ | 0 | 0-± | 1-2+ C | NS | 0 | 0-1+ M, C | NS | May 15, 2003 |
| INES01710A | Esophagus | Esophagus | 0 | 0 | 0 | 100 C | 0 | 0 | 2+ C | NS | 0 | 0-2+ M, C | NS | May 15, 2003 |
| INES01770A | Esophagus | Esophagus | 0 | 0 | 0 | 100 C | 0 | ± | 3+ C³ | NS | 0 | 0 | NS | May 15, 2003 |
| INES01772A | Esophagus | Esophagus | 0 | 0 | 0 | 100 C | 0 | 0 | 1-2+ C | NS | 0 | NS | NS | May 15, 2003 |
| INHE01517A | Heart | Heart | 0 | 0 | 0 | 100 C | 0 | 0 | 2+ C | NS | 0 | NS | NS | May 15, 2003 |
| INHE01520A | Heart | Heart | 0 | 0 | 0 | 0 | 100 | 0 | 2+ C | NS | 0 | NS | NS | May 15, 2003 |
| INHE05535A | Heart | Heart | 0 | 0 | 0 | 100 C | 0 | ±-1+ C | 2+ C | NS | 0 | NS | NS | May 30, 2003 |
| INKI00956A | Kidney | Kidney | 0 | 90 C¹ | 0 | 0 | 10 | 0-1+ | NS | NS | 0 | NS | NS | May 30, 2003 |
| INKI00323J | Kidney | Kidney | 0 | 50 C¹ | 30 C¹ | 10 C¹ | 10 | ± | 2+ C | NS | 0 | 0 | NS | May 30, 2003 |
| INKI00968A | Kidney | Kidney | 0 | 30 C¹ | 10 C¹ | 50 C¹ | 10 | ±-1+ | 2+ C | NS | 0 | 0 | NS | May 30, 2003 |
| INLI02224A | Liver | Liver | 0 | 0 | 0 | 80 C¹ | 20 | 1+ C | 1+ C | NS | 0 | 0-3+ M, C | NS | May 6, 2003 |
| INLI03366A | Liver | Liver | 0 | 0 | 0 | 100 C¹ | 0 | ± | 1+ C | NS | 0 | 0-3+ M, C | NS | May 6, 2003 |
| INLI03372A | Liver | Liver | 0 | 0 | 0 | 60 C¹ | 40 | ± | 1+ C | NS | 0 | 0-3+ M, C | NS | May 6, 2003 |
| INLU02148A | Lung | Lung | 0 | 0 | 0 | 100 C | 0 | 1+ C | 1+ C | NS | 0-± | 0-3+ M, C | NS | May 6, 2003 |
| INLU01551A | Lung | Lung | 0 | 0 | 0 | 80 C | 20 | 1+ C | 1+ C | NS | 0-± | 0-3+ C | NS | May 6, 2003 |
| INLU01506A | Lung | Lung | 0-± | 0 | 10 C | 90 C | 0 | 1+ C | 1+ C | NS | ± | 0-3+ M, C | NS | May 6, 2003 |
| INLN02925A | Lymph Node | Lymph Node | 0 | 5 M, C | 5 M, C | 20 C⁵ | 70 | 2+ M, C | 1-2+ C | NS | ± | NS | NS | May 6, 2003 |
| INLN01624D | Lymph Node | Lymph Node | 0 | 1 M, C | 1 M, C | 10 C⁵ | 88 | ± | 1+ C | NS | 0-± | NS | NS | May 6, 2003 |
| INLN01620A | Lymph Node | Lymph Node | 0 | 5 M, C | 3 M, C | 20 C⁵ | 72 | 3+ M | 2+ C | NS | ±-1+ F | NS | NS | May 6, 2003 |
| INMU01512A | Muscle | Skeletal Muscle | 0 | 0 | 0 | 50 C | 50 | 1+ C | 1+ C | NS | ± | NS | NS | May 6, 2003 |
| INMU01513A | Muscle | Skeletal Muscle | 0 | 0 | 0 | 0 | 100 | 2-3+ M, C | 1-2+ C, F | NS | NS | NS | NS | May 6, 2003 |
| INMU0110-307-00822-8 | Muscle | Skeletal Muscle | 0 | 0 | 0 | 0 | 100 | 2+ M, F | 1-2+ C, F | NS | 0 | NS | NS | May 6, 2003 |
| INOV04286A | Ovary | Ovary | 0 | 0 | 0 | 100 C | 0 | 3+ M | 1-2+ C, F | NS | ± | NS | NS | May 6, 2003 |
| INOV04320A | Ovary | Ovary | 0 | 0 | 0 | 100 C | 0 | 0-± | 1+ C | NS | ± | NS | NS | May 6, 2003 |
| INOV04321A | Ovary | Ovary | 0 | 0 | 0 | 100 C | 0 | 2-3+ M | 1-2+ C | NS | ± | NS | NS | May 6, 2003 |
| INPN01215A | Pancreas | Pancreas | 0-3+ C, F⁶ | 0 | 50 C¹ | 50 C¹ | 0 | 1+ C | 1-2+ C, F | NS | ± | 0-3+ C, M | 0 | May 6, 2003 |
| INPN01503A | Pancreas | Pancreas | 0-3+ C, F⁶ | 0 | 60 C¹ | 40 C¹ | 0 | ± | 1+ C | NS | 0-± | NS | NS | May 6, 2003 |
| INPN0203-307-00245-2 | Pancreas | Pancreas | 0-3+ C, F | 0 | 40 C¹ | 60 C¹ | 0 | 1+ C | 1+ C | NS | 0 | NS | NS | May 6, 2003 |
| INPO04089A | Parotid | Parotid | 0-1+ C, F⁷ | 0 | 10 C^{7, 1} | 50 C¹ | 40 | 3+ M, C | 1+ C | NS | 0-± | NS | 0-± | May 6, 2003 |
| INPO0105-303-0818-6A | Parotid | Parotid | 0-1+ C, F⁷ | 0 | 10 C^{7, 1} | 0 | 90 | 3+ M, C | 1+ C, F | NS | 0-± | 0-3+ M, C | NS | May 6, 2003 |
| INPO0109-192-00553-1 | Parotid | Parotid | 0-1+ C, F⁷ | 0 | 20 C¹ | 80 C¹ | 0 | 0-± | 1+ C | NS | 0-± | 0-3+ M, C | NS | May 6, 2003 |
| INPI03653A | Pituitary | Pituitary | 0-3+ C⁸ | 0 | 1 C | 99C | 0 | 1-2+ C | 1-2+ C | NS | ±-1+ F | NS | NS | May 6, 2003 |
| INPI03655A | Pituitary | Pituitary | 0-2+ C⁸ | 0 | 0 | 100 C | 0 | 0 | 1+ C | NS | ± | NS | NS | May 6, 2003 |
| INPI03656A | Pituitary | Pituitary | 0-3+ C, F⁵ | 0 | 20 C | 80 C | 0 | 2+ C | NS | NS | ± | NS | NS | May 6, 2003 |
| INPL03550A | Placenta | Placenta | 0 | 90 M, C | 10 M, C | 0 | 0 | 1-2+ C | 0-1+ C | NS | 0-± | NS | NS | May 6, 2003 |
| INPL03554A | Placenta | Placenta | 0 | 80 M, C | 20 M, C | 0 | 0 | 2+ M, C | 1+ C | NS | ± | NS | NS | May 6, 2003 |
| INPL03555A | Placenta | Placenta | 0 | 70 M, C | 30 M, C | 0 | 0 | 2- | 1+ C | NS | 0-± | NS | NS | May 6, 2003 |
| | | | | | | | | 3 M, C | | | | | | |
| IBPR00935A | Prostate | Prostate | 0 | 0 | 30 C | 70 C | 0 | 1-2+ | 1+ C | NS | ± | NS | NS | May 6, 2003 |
| INPR02945A | Prostate | Prostate | 0 | 0 | 50 C | 50 C | 0 | 3+ M, C | 1+ C | NS | ±-1+ | 3+ M, C | NS | May 6, 2003 |
| INPR02960A | Prostate | Prostate | 0 | 0 | 40 C | 60 C | 0 | 3+ M, C | 1+ C | NS | 0-1+ F | 3+ M, C | NS | May 6, 2003 |
| INSK01361L | Skin | Skin | 0 | 0 | 0 | 100 C | 0 | 1+ C | NS | NS | 0 | 0 | NS | May 30, 2003 |
| INSK01342B | Skin | Skin | 0 | 0 | 0 | 100 C | 0 | ± | 3+ C | NS | 0 | NS | NS | May 30, 2003 |
| INSK01339T | Skin | Skin | 0 | 0 | 0 | 100 C | 0 | NS | 2+ C | NS | 0 | NS | NS | May 30, 2003 |
| INSM03230A | Small Intestine | Small Intestine | 0 | 0 | 0 | 100⁹ | 0 | 0 | 2+ C | NS | 0 | NS | NS | May 15, 2003 |
| INDU00182B | Small Intestine | Small Intestine | 0-± | 0 | 15C¹ | 85C¹ | 0 | 0 | 1-2+ C | NS | 0 | 0-1+ C | NS | May 15, 2003 |
| INJE00181B | Small Intestine | Small Intestine | 0 | 0 | 0 | 100 C | 0 | 0 | 1-2+ C | NS | 0 | 0-3+ C, M | NS | May 15, 2003 |
| INSC03662A | Spinal Cord | Spinal Cord | 0 | 0 | 0 | 100 C | 0 | ± | 2+ C | NS | 0 | 0-1+ C | NS | May 15, 2003 |
| INSC03664A | Spinal Cord | Spinal Cord | 0 | 0 | 0 | 0 | 100 | 0 | 2+ C | NS | 0 | NS | 0-± | May 15, 2003 |
| INSC03666A | Spinal Cord | Spinal Cord | 0 | 0 | 0 | 0 | 100 | 0 | 2+ C | NS | ±-1+² | NS | 0 | May 15, 2003 |
| INSP02772B | Spleen | Spleen | 0 | 0 | 0 | 0 | 100 | 0 | NS | NS | 0-1² | NS | NS | May 15, 2003 |
| INSP03695A | Spleen | Spleen | 0¹⁰ | 0 | 0 | 0 | 100 | 0 | 2+ C | NS | 0-1+ | NS | NS | May 15, 2003 |
| INSP03699A | Spleen | Spleen | 0 | 0 | 0 | 0 | 100 | 0 | 1+ C | NS | 0-± | NS | NS | May 30, 2003 |
| INST02911B | Stomach | Stomach | 0 | 0 | 0 | 0 | 100 | 0 | 1-2+ C | NS | 0 | NS | NS | May 15, 2003 |
| INST03697A | Stomach | Stomach | 0 | 0 | 20 C¹ | 80 C¹ | 0 | 0 | 1-2+ C | NS | 0 | 0-3+ C | NS | May 15, 2003 |
| INST0103-212-01588-1 | Stomach | Stomach | 0 | 0 | 35C¹ | 65C¹ | 0 | 0 | 1-2+ C | NS | 0 | 0-1+ | NS | May 15, 2003 |
| INTE04514A | Testis | Testis | 0 | 0 | 80 C | 20 C | 0 | 0 | 1+ C | NS | 0 | NS | NS | May 15, 2003 |
| INTE04516A | Testis | Testis | 0 | 0 | 0 | 90 C | 10 | 0 | 1+ C | NS | 0 | NS | NS | May 15, 2003 |
| INTE04517A | Testis | Testis | 0 | 0 | 80 C | 20 C | 0 | NS | NS | NS | 0 | NS | NS | May 15, 2003 |
| INTH03667A | Thyroid | c/w Thyroid | 0-± | 3 C¹ | 2 C¹ | 0 | 95 | 0 | 1+ C | NS | 0 | 0-3+ M, C | NS | May 15, 2003 |
| INTH03669A | Thyroid | Thyroid | 0 | 0 | 0 | 0 | 100 | 0 | 2+ C | NS | 0 | NS | NS | May 15, 2003 |
| INTH03671A | Thyroid | Thyroid | 0 | 2 C¹ | 0 | 98C¹ | 0 | 0 | 2+ C | NS | ± | NS | NS | May 15, 2003 |
| INTO03508A | Tonsil | Tonsil | 0 | 0 | 0 | 0 | 100 | 1+ C | NS | NS | ± | NS | NS | May 30, 2003 |
| INTO03507A | Tonsil | Tonsil | 0 | 0 | 0 | 0 | 100 | 1+ | NS | NS | 0 | NS | NS | May 30, 2003 |
| INTO03449A | Tonsil | Tonsil | 0 | 5 M, C | 5 M, C | 10 C | 80 | 0 | 2+ C | NS | 0 | NS | NS | May 30, 2003 |
| INUR03677A | Ureter | Ureter | 0 | 0 | 90 C¹ | 10 C¹ | 0 | ± | 2+ C | NS | 0-± | NS | NS | May 15, 2003 |
| INUR03685B | Ureter | Ureter | 0 | 0 | 0 | 100 C¹ | 0 | 0 | 2+ C | NS | 0 | NS | NS | May 15, 2003 |
| INUR05530A | Ureter | Ureter | 0 | 0 | 0 | 100 C | 0 | 0-± | 2+ C | NS | 0 | NS | NS | May 30, 2003 |
| INUT01842A | Uterus | Uterus | 0 | 0 | 0 | 100 C¹ | 0 | 0 | 1-2+ C | NS | 0 | NS | NS | May 15, 2003 |
| INUT0105-306-0054-11 | Uterus | Uterus | 0 | 0 | 0 | 100 C¹ | 0 | 0 | 1-2+ C | NS | 0 | NS | NS | May 15, 2003 |
| INUT0103-212-01579-2 | Uterus | Uterus | 0 | 0 | 25C¹ | 75C¹ | 0 | 0 | 1+ C | NS | 0 | NS | NS | May 15, 2003 |

Comments/Notes:
¹Cytoplasmic granular staining of undetermined significance
²Neuropil
³Diffuse precipitate not cell-associated (between cells)
⁴Endocervical epithelium show membrane staining; squamous epithelium show cytoplasmic, granular staining.
⁵Sinus histiocytes are 1+ C.
⁶Rare strongly positive cells of undetermind significance
⁷Ductal epithelium is positive.
⁸Rare, (<1%) scattered positive cells
⁹Weak staining only
¹⁰Scattered hemosiderin pigment present in tissue sample
± = Equivocal Results
NA = Not Applicable
NS = Not Seen
Ap = Apical Staining
B = Basal Layer Staining
C = Cytoplasmic Staining
F = Focally Positive
c/w = Consistent With
H = Heterogeneous Staining
I = Inflammatory Cells
La = Luminal Accentuation
M = Membrane Staining
N = Nuclear Staining
P = Perincural Staining
S = Stroma
Sc = Scattered

### EXAMPLE 6

This example demonstrates the precision of the IL13-Rα2 antibody described in Example 1.

Precision analysis was performed on 3 human astrocytoma specimens and 1 human renal cell carcinoma specimen on five different days under the same test conditions. The immunohistochemistry was performed as described in Example 4. Adequate precision was demonstrated by a generally consistent staining pattern and intensity of the tissues tested on five different days (See Table 3).

TABLE 3
Antibody Validation
Precision Worksheet

| | |
|---|---|
| Study No.: | NPH13 |
| Antibody: | Affi-Anti-IL-13Rα2 IgY |
| Clone: | Polyclonal, Affinity Purified |
| Manufacturer: | GenWay Biotech, Inc. |
| Target: | IL-13Rα2 |
| Working Conc.: | 10 μg/ml |
| Pretreatment: | HIER (BORG) 120° C. 3′ followed by trypsin 1′ |
| Isotype: | Chicken IgY |

| | | | | Staining of | | | | |
|---|---|---|---|---|---|---|---|---|
| General Specimen Information | | | | Distinctive Tissue Elements | | | | |
| | | | Isotype | % Cells | | | | |
| | | | Control | Staining at Each Intensity | | | | Date |
| Specimen ID# | Tissue Type | Histological Review | Background | 3+ | 2+ | 1+ | 0 | Staining |

Day 1

| | | | | | | | | |
|---|---|---|---|---|---|---|---|---|
| ITKI02476B | Kidney CA | Kidney CA | 0 | 20 C, M | 40 C, M | 40 C, M | 0 | May 15, 2003 |
| ITCC04367D | A172 Cell Line | Cell Line | 0 | 0 | 20 C | 80 C | 0 | May 15, 2003 |
| ITGL0110-307-02312-1 | Glioblastoma | c/w Glioblastoma | 0-± | 15 C, M | 40 C | 35 C | 10 | May 15, 2003 |
| ITGL0110-307-02305-1 | Glioblastoma | c/w Glioblastoma | 0-± | 0 | 0 | 90 C | 10 | May 15, 2003 |
| ITGL0110-307-02285-1 | Glioblastoma | c/w Glioblastoma | 0 | 0 | 30 C | 70 C | 0 | May 15, 2003 |
| ITKI02473A | Renal Cell CA | c/w Renal Cell CA | ± | 30 M, C | 30 M, C | 30 C | 10 | May 15, 2003 |

Day 2

| | | | | | | | | |
|---|---|---|---|---|---|---|---|---|
| ITKI02476B | Kidney CA | Kidney CA | 0 | 20 C, M | 50 C, M | 30 C | 0 | May 20, 2003 |
| ITCC04367D | A172 Cell Line | Cell Line | 0 | 5 C | 10 C | 85 C | 0 | May 20, 2003 |
| ITGL0110-307-02312-1 | Glioblastoma | c/w Glioblastoma | 0 | 5 C | 30 C | 65 C | 0 | May 20, 2003 |
| ITGL0110-307-02305-1 | Glioblastoma | c/w Glioblastoma | 0 | 20 C, M | 10 C | 10 C | 60 | May 20, 2003 |
| ITGL0110-307-02285-1 | Glioblastoma | c/w Glioblastoma | 0 | 2 C | 70 C | 28 C | 0 | May 20, 2003 |
| ITKI02473A | Kidney CA | Kidney CA | 0 | 10 C, M | 10 C, M | 80 M, C | 0 | May 20, 2003 |

Day 3

| | | | | | | | | |
|---|---|---|---|---|---|---|---|---|
| ITKI02476A | Kidney CA | Kidney CA | 0 | 30 C, M | 40 C, M | 30 C, M | 0 | May 30, 2003 |
| ITCC04367D | A172 Cell Line | Cell Line | 0 | 0 | 60 C, M | 40 C, M | 0 | May 30, 2003 |
| ITGL0110-307-02312-1 | Glioblastoma | c/w Glioblastoma | 0 | 10 C | 40 C | 50 C | 0 | May 30, 2003 |
| ITGL0110-307-02305-1 | Glioblastoma | c/w Glioblastoma | 0 | 0 | 10 C | 80 C | 10 | May 30, 2003 |
| ITGL0110-307-02285-1 | Glioblastoma | c/w Glioblastoma | 0 | 0 | 20 C | 80 C | 0 | May 30, 2003 |
| ITKI02473A | Kidney CA | c/w Renal Cell CA | 0 | 20 M, C | 40 M, C | 30 M, C | 10 | May 30, 2003 |

Day 4

| | | | | | | | | |
|---|---|---|---|---|---|---|---|---|
| ITKI02476A | Kidney CA | Kidney CA | 0 | 30 C, M | 30 C, M | 40 C, M | 0 | Jun. 2, 2003 |
| ITCC04367D | A172 Cell Line | Cell Line | 0 | 0 | 10 C | 90 C | 0 | Jun. 2, 2003 |
| ITGL0110-307-02312-1 | Glioblastoma | c/w Glioblastoma | 0 | 10 C | 30 C | 50 C | 10 | Jun. 2, 2003 |
| ITGL0110-307-02305-1 | Glioblastoma | c/w Glioblastoma | 0 | 0 | 20 C | 80 C | 0 | Jun. 2, 2003 |
| ITGL0110-307-02285-1 | Glioblastoma | c/w Glioblastoma | 0 | 0 | 5 C | 95 C | 0 | Jun. 2, 2003 |
| ITKI02473A | Kidney CA | c/w Kidney CA | 0 | 20 C | 40 C | 30 C | 10 | Jun. 2, 2003 |

Day 5

| | | | | | | | | |
|---|---|---|---|---|---|---|---|---|
| ITKI02476B | Kidney CA | Renal Cell CA | 0 | 50 C, M | 20 M, C | 30 C, M | 0 | Jun. 10, 2003 |
| ITCC04367D | A172 Cell Line | Cell Line | 0-1+ C | 0 | 50 C | 50 C | 0 | Jun. 10, 2003 |
| ITGL0110-307-02312-1 | Glioblastoma | c/w Glioblastoma | 0 | 30 C | 20 C | 40 C | 10 | Jun. 10, 2003 |
| ITGL0110-307-02305-1 | Glioblastoma | c/w Glioblastoma | 0 | 40 C | 20 C | 40 C | 0 | Jun. 10, 2003 |
| ITGL0110-307-02285-1 | Glioblastoma | c/w Glioblastoma | 0 | 0 | 10 C | 90 C | 0 | Jun. 10, 2003 |
| ITKI02473A | Kidney CA | c/w Kidney CA | 0 | 30 C | 40 C | 30 C | 0 | Jun. 10, 2003 |

Comments/Notes:
± = Equivocal Results
NA = Not Applicable
NS = Not Seen
Ap = Apical Staining
B = Basal Layer Staining
C = Cytoplasmic Staining
F = Focally Positive
c/w = Consistens With
H = Heterogeneous Staining
I = Inflammatory Cells
La = Luminal Accentuation
M = Membrane Staining
N = Nuclear Staining
P = Perineural Staining
S = Stroma
Sc = Scattered

### EXAMPLE 7

This example compares RT-PCR to IHC using the IL13-Rα2 antibody described in Example 1.

A method comparison study, comparing RT-PCR to immunohistochemistry, was performed on 5 formalin-fixed, paraffin-embedded human glioblastoma specimens with adjacent tumor preserved for RNA extraction. The immunohistochemistry was performed as described in Example 4. Following RNA extraction, quantitative RT-PCR was performed on each specimen to determine the expression of the IL13-Rα2 gene. It is expected that IL13-Rα2 message will be presented in specimens that exhibit positive IHC staining.

Adequate method comparison was demonstrated by a qualitative agreement between the IHC staining pattern and intensity and the RT-PCR gene expression of the specimens. (See Table 4). Although there appears to be a general concordance between the two methods, a quantitative comparison between the results of the two methods does not show a high level of correlation. Factors to be considered in such a comparison include 1) the level of IL13-Rα2 mRNA may not directly correlate with protein expression, 2) the adjacent tumor specimens may have a different density of tumor cells, and 3) while RT-PCR is a true scalar quantity, immunohistochemistry is, at best, semi-quantitative. Considering all the factors, the method comparison of IL13-Rα2 expression between RT-PCR and IHC appears adequate.

TABLE 4
Antibody Validation
Method Comparison Study

| | | | | IHC Staining of Distinctive | | | | RT-PCR Results² | |
|---|---|---|---|---|---|---|---|---|---|
| General Specimen Information | | | | Tissue Elements¹ | | | | Mean Quantity | Mean Quantity |
| | | | Isotype | % Cells | | | | IL-13Rα2 | Actin |
| | | Histological | Control | Staining at Each Intensity | | | | (copy #)/50 ng | (copy #)/50 ng |
| Specimen ID# | Tissue Type | Review | Background | 3+ | 2+ | 1+ | 0 | RNA | RNA |
| ITKI02476B | Kidney CA | Kidney CA | 0 | 20 C, M | 50 C, M | 30 C | 0 | NA | NA |
| ITCC04367D | A172 Cell Line | Cell Line | 0 | 5 C | 10 C | 85 C | 0 | NA | NA |
| 02-703306 | Brain Tumor | c/w Glioblastoma | 2+ C | 90 C | 10 C | 0 | 0 | 8103.30 | 1822.35 |
| 03-701458 | Brain Tumor | c/w Glioblastoma | 1+ C | 40 C | 20 C | 25 C | 15 | 6056.49 | 933.15 |
| 03-701460 | Brain Tumor | c/w Glioma | 0-1+ F | 20 M, C | 20 M, C | 30 C, M | 30 | 1877.30 | 476.85 |
| 03-701462 | Brain Tumor | c/w Mixed Glioma | 0-2+ C | 30 M, C | 20 M, C | 10 C | 40 | 1037.51 | 5140.10 |
| 03-701411 | Brain Tumor | c/w Glioblastoma | 0-2+ C | 60 C, M | 20 C, M | 20 C, M | 0 | 2303.73 | 7364.55 |

± = Equivocal Results
NA = Not Applicable
NS = Not Seen
Ap = Apical Staining
B = Basal Layer Staining
C = Cytoplasmic Staining
F = Focally Positive
c/w = Consistent With
H = Heterogencous Staining
I = Inflammatory Cells
La = Luminal Accentuation
M = Membrane Staining
N = Nuclear Staining
P = Perineural Staining
S = Stroma
Sc = Scattered

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein, including the following:
Bernard et al., Lab. Invest., 81(9), 1223 (2001)
Daines et al., J. Biol. Chem., 277(12), 10387-93 (2002)
Daput et al., J. Biol. Chem., 271(28), 16921-26 (1996)
David et al., Oncogene, 20(46), 6660-68 (2001)
Debinski et al., Clin. Canc. Res., 5, 985-90 (1999)
Donaldson et al., J. Immunol., 161, 2317-24 (1998)
Feng et al., Lab. Invest., 78(5), 591-602 (1998)
Joshi et al., Canc. Res., 60, 1168-72 (2000)
Joshi et al., Clin. Canc. Rres., 8(6), 1948-56 (2002)
Kawakarni et al., Blood, 97(9), 2673-79 (2001)
Kawakami et al., Int. J. Cancer, 103(1), 45-52 (2003)
Kawakami et al., J. Immunol., 169(12), 7119-26 (2002)
Liu et al., Cancer Immunol. Immunother., 49(6), 319-424 (2000)
Maini et al., J. Urol., 158 (3Pt1), 948-53 (1997)
Rahaman et al., Canc. Res., 62, 11-3-09 (2002)
Tang et al., Vet. Immunol. Immunopathol., 79(3-4), 181-95 (2001)
Vita et al., J. Biol. Chem., 270(8), 3512-17 (1995)
Wu et al., J. Neurooncol., 59(2), 99-105 (2002)

What is claimed is:

1. An isolated antibody or antigen-binding fragment thereof directed against an interleukin 13 receptor alpha 2 (IL13-Rα2) that binds an epitope consisting essentially of an amino acid sequence of SEQ ID NO:1.

2. The antibody of claim 1, wherein the epitope is encoded by a nucleic acid sequence consisting essentially of SEQ ID NO:2.

3. The antibody of claim 1, wherein the antibody is a monoclonal antibody.

4. The antibody of claim 1, wherein the antibody is selected from a group consisting of a chicken antibody, a mouse antibody, a human antibody, and a humanized antibody.

5. The antibody of claim 1, wherein the antibody is a Fab fragment, an F(ab′)2 fragment or a single chain Fv fragment.

6. The antibody of any of claims 1-5, wherein the antibody is conjugated to a cytotoxic agent.

7. The antibody of claim 6, wherein the cytotoxic agent is selected from a group consisting of Pseudomonas exotoxin, Diphtheria toxin, abrin, and a radionuclide.

8. The antibody of claim 1, further comprising a physiologically acceptable carrier.

9. A method for detecting the presence of an IL13-Rα2 polypeptide comprising (a) contacting a cell suspected of containing IL13-Rα2 with an isolated antibody or fragment thereof that binds to IL13-Rα2, and (b) detecting the binding of the IL13-Rα2 antibody to IL13-Rα2.

10. A method for localizing IL13-Rα2 in a sample or cell comprising (a) contacting the sample or cell with an isolated antibody or fragment thereof that binds to IL13-Rα2, (b) detecting binding of the antibody to the IL13-Rα2, and (c) determining the location of the IL13-Rα2 in the sample or cell.

11. A method for diagnosing a disease characterized by the expression of an IL13-Rα2 comprising contacting a sample or cell with an isolated antibody or fragment thereof that binds to IL13-Rα2, wherein the detectable binding of the IL13-Rα2 antibody indicates expression of the IL13-Rα2, and the disease is diagnosed.

12. A method for killing a cell that expresses IL13-Rα2 comprising contacting the cell with an isolated antibody or fragment thereof that binds to the IL13-Rα2 and is conjugated to a cytotoxic agent, such that the IL13-Rα2 antibody binds to IL13-Rα2 and the cytotoxic agent contacts the cell, whereby the cell is killed.

13. The method of any of claims 9-12, wherein the antibody binds the epitope consisting essentially of an amino acid sequence of SEQ ID NO:1.

14. The method of any of claims 9-12, wherein the antibody is a monoclonal antibody.

15. The method of any of claims 9-12, wherein the antibody is selected from a group consisting of a chicken antibody, a mouse antibody, a human antibody, and a humanized antibody.

16. The method of claim 9, wherein the cell is in vitro.

17. The method of claim 9, wherein the cell is in vivo.

18. The method of any of claims 9 or 11, wherein the detecting is by flow cytometry, enzyme linked immunosorbent assay (ELISA), affinity chromatography, competitive inhibition assay, radioimmunoassay, immunoflorescence microscopy, immunoelectron microscopy, immunocytochemistry, or immunoprecipitation.

19. The method of claim 10, wherein the detecting and determining are by immunoflorescence microscopy, immunoelectron microscopy, or immunocytochemistry.

20. The method of claim 12, wherein the cytotoxic agent is selected from a group consisting of Pseudomonas exotoxin, Diphtheria toxin, abrin, and a radionuclide.

21. The method of claim 12, wherein the contacting comprises administering the IL13-Rα2 antibody to a human.

22. The method of claim 21, wherein the administration is intravenous, intraperitoneal, or intratumoral.

23. The method of any of claims 11 or 12, wherein the cell is a tumor cell.

24. The method of claim 23, wherein the tumor cell is a malignant glioma cell.


SEQUENCE LISTING (4 sequences)

SEQ ID NO: 1 - protein, 310 aa, Artificial
  Synthetic
SDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGED

SEQ ID NO: 2 - DNA, 930 bp, Artificial
  Synthetic
tcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagac

SEQ ID NO: 3 - DNA, 1119 bp, Homo sapiens
  misc_feature: Interleukin 13 receptor, alpha 2
atggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgatt

SEQ ID NO: 4 - protein, 380 aa, Homo sapiens
  MISC_FEATURE: Interleukin 13 receptor, alpha 2
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT